(19) United States
(12) Patent Application Publication
(10) Pub. No.: US 2026/0077158 A1
(43) Pub. Date: Mar. 19, 2026

(54) CATHETER SHAFT AND METHODS FOR MANUFACTURING CATHETER SHAFT FROM A PLURALITY OF DISCRETE SHAFT SEGMENTS

(71) Applicant: BOSTON SCIENTIFIC SCIMED, INC., Maple Grove, MN (US)

(72) Inventors: David John Onushko, Maple Grove, MN (US); Daniel James Viola, Minneapolis, MN (US); Sara Reda Eskander, Plymouth, MN (US); Carly Rogers, Eden Prairie, MN (US)

(73) Assignee: BOSTON SCIENTIFIC SCIMED, INC., Maple Grove, MN (US)

(21) Appl. No.: 19/323,117

(22) Filed: Sep. 9, 2025

Related U.S. Application Data
(60) Provisional application No. 63/694,392, filed on Sep. 13, 2024.

(51) Int. Cl. A61M 25/00

(52) U.S. Cl. CPC A61M 25/0052; A61M 25/0012; A61M 25/0045; A61M 2205/32; A61M 2207/00

(57) ABSTRACT

Medical devices such as catheter shafts as well as methods for manufacturing catheters shafts are disclosed. An example method for manufacturing a catheter shaft may include forming a first shaft member. The first shaft member may include a first inner layer, a first reinforcing layer, and a first outer layer. The method may also include forming a second shaft member. The second shaft member may include a second inner layer, a second reinforcing layer, and a second outer layer. The method may also include trimming the first shaft member to form a first shaft segment, trimming the second shaft member to form a second shaft segment, and securing the first shaft segment to the second shaft segment.

### CROSS REFERENCE TO RELATED APPLICATIONS

The present application claims the benefit of U.S. Provisional Patent Application Ser. No. 63/694,392, filed Sep. 13, 2024, the disclosure of which is incorporated herein by reference.

### TECHNICAL FIELD

The present disclosure pertains to medical devices, and methods for manufacturing medical devices. More particularly, the present disclosure pertains to a catheter shaft and methods for the manufacturing catheter shaft from a plurality of discrete shaft segments.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A method for manufacturing a catheter shaft is disclosed. The method comprises: forming a first shaft member, the first shaft member including a first inner layer, a first reinforcing layer, and a first outer layer; forming a second shaft member, the second shaft member including a second inner layer, a second reinforcing layer, and a second outer layer; wherein at least one of (a) the first inner layer differs from the second inner layer, (b) the first reinforcing layer differs from the second reinforcing layer, and (c) the first outer layer differs from the second outer layer; trimming the first shaft member to form a first shaft segment; trimming the second shaft member to form a second shaft segment; and securing the first shaft segment to the second shaft segment.

Alternatively or additionally to any of the embodiments above, the first inner layer is radially thicker than the second inner layer.

Alternatively or additionally to any of the embodiments above, the first reinforcing layer includes a first braid and wherein the second reinforcing layer includes a second braid different from the first braid.

Alternatively or additionally to any of the embodiments above, the first reinforcing layer includes a braid and wherein the second reinforcing layer includes a coil.

Alternatively or additionally to any of the embodiments above, the first reinforcing layer includes a metal and wherein the second reinforcing layer includes a polymer.

Alternatively or additionally to any of the embodiments above, the first reinforcing layer is radially offset from the second reinforcing layer.

Alternatively or additionally to any of the embodiments above, the first outer layer is radially thicker than the second outer layer.

Alternatively or additionally to any of the embodiments above, securing the first shaft segment to the second shaft segment includes securing the first shaft segment to the second shaft segment with an adhesive bond.

Alternatively or additionally to any of the embodiments above, securing the first shaft segment to the second shaft segment includes securing the first shaft segment to the second shaft segment with a thermal bond.

Alternatively or additionally to any of the embodiments above, securing the first shaft segment to the second shaft segment includes securing the first shaft segment to the second shaft segment with a joining structure.

A medical device is disclosed. The medical device comprises: an elongate shaft formed from a plurality of discrete shaft segments that are joined together, the plurality of discrete shaft segments including a first shaft segment and a second shaft segment; wherein the first shaft segment includes a first inner layer, a first reinforcing layer, and a first outer layer; wherein the second shaft segment includes a second inner layer, a second reinforcing layer, and a second outer layer; and wherein at least one of (a) the first inner layer differs from the second inner layer, (b) the first reinforcing layer differs from the second reinforcing layer, and (c) the first outer layer differs from the second outer layer.

Alternatively or additionally to any of the embodiments above, the first reinforcing layer includes a first braid and wherein the second reinforcing layer includes a second braid different from the first braid.

Alternatively or additionally to any of the embodiments above, the first reinforcing layer includes a braid and wherein the second reinforcing layer includes a coil.

Alternatively or additionally to any of the embodiments above, the first reinforcing layer includes a metal and wherein the second reinforcing layer includes a polymer.

Alternatively or additionally to any of the embodiments above, the first reinforcing layer includes a tungsten braid and wherein the second reinforcing layer includes a stainless steel braid.

Alternatively or additionally to any of the embodiments above, the first reinforcing layer is radially offset from the second reinforcing layer.

Alternatively or additionally to any of the embodiments above, further comprising a third shaft segment coupled to the second shaft segment.

Alternatively or additionally to any of the embodiments above, the third shaft segment is free of a reinforcing layer.

Alternatively or additionally to any of the embodiments above, the third shaft segment includes a radiopaque marker.

A method for manufacturing a catheter shaft is disclosed. The method comprises: forming a first shaft segment, the first shaft segment including a first inner layer, a first outer layer, and a first reinforcing member disposed between the first inner layer and the first outer layer; forming a second shaft segment, the second shaft segment including a second inner layer, a second outer layer, and a second reinforcing member disposed between the second inner layer and the second outer layer; wherein at least one of (a) the first inner layer differs from the second inner layer, (b) the first outer layer differs from the second outer layer, and (c) the first reinforcing member differs from the second reinforcing member; trimming a first pre-determined length of the first shaft segment to form a first shaft segment; trimming a second pre-determined length of the second shaft segment to form a second shaft segment; and securing the first shaft segment to the second shaft segment.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example delivery system.

FIG. 2 is a side view of an example delivery system.

FIGS. 3A-3B are cross-sectional side views of a portion of an example medical device.

FIGS. 4A-4B are cross-sectional side views of a portion of an example medical device.

FIGS. 5A-5B are cross-sectional side views of a portion of an example medical device.

FIGS. 6A-6B are cross-sectional side views of a portion of an example medical device.

FIG. 7 is a cross-sectional side view of a portion of an example medical device.

FIG. 8 is a side view of an example delivery system.

FIG. 9 is a side view of an example delivery system.

FIG. 10 is a side view of an example delivery system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

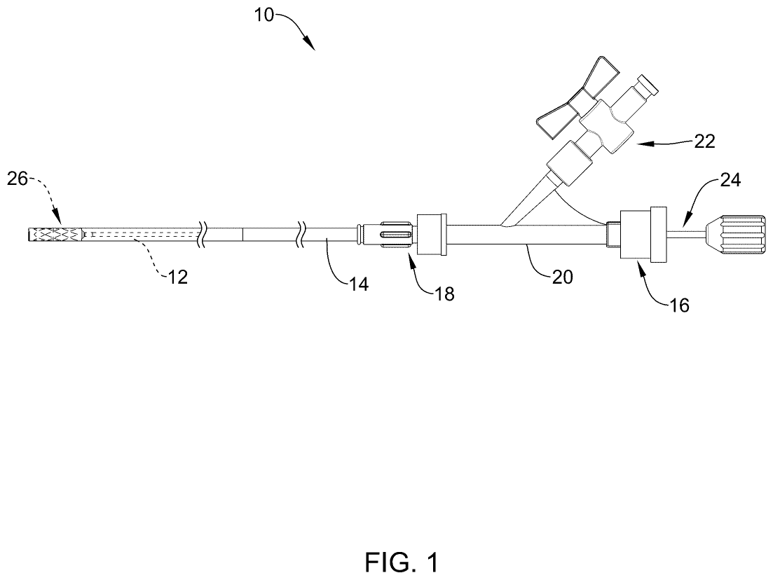

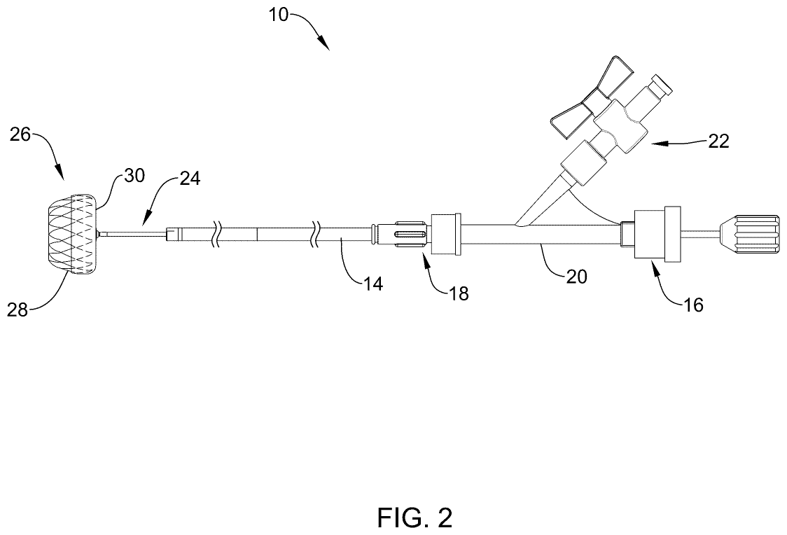

FIGS. 1-2 schematically illustrate selected components and/or arrangements of an occlusive implant system. It should be noted that in any given figure, some features of the occlusive implant system may not be shown, or may be shown schematically, for simplicity. Additional details regarding some of the components of the occlusive implant system may be illustrated in other figures in greater detail. The occlusive implant system may be used to deliver and/or deploy a variety of medical implants (e.g., a cardiovascular implant, an occlusive implant, etc.) to one or more locations within the anatomy, including but not limited to, in some embodiments, the heart and/or the left atrial appendage. In the interest of clarity, the following discussion refers to an occlusive implant, but other medical implants may be used and/or considered with the occlusive implant system.

The occlusive implant system may include a delivery system 10 including a delivery sheath 14 having a delivery lumen 12 extending proximally from a distal end of the delivery sheath 14. In one example, the delivery lumen 12 extends from a proximal opening to a distal opening of the delivery sheath 14. The delivery system 10 may include a proximal hub 16. In some embodiments, the delivery system may include a mid-hub 18. In some embodiments, the delivery system 10 may include a mid-shaft 20 extending from the proximal hub 16 to the mid-hub 18. In some embodiments, the delivery sheath 14 may extend distally from the mid-hub 18. Other configurations are also contemplated. In some embodiments, the delivery system 10 may include a side port 22. In some embodiments, the side port 22 may be in communication with the mid-shaft 20. Other configurations are also contemplated. In some embodiments, the delivery system 10 and/or the delivery lumen 12 may include a proximal segment (not shown) extending within and/or through the mid-hub 18, the mid-shaft 20, and the proximal hub 16. In some embodiments, the proximal segment may be in fluid communication with and/or may be an extension of the delivery lumen 12 of the delivery sheath 14. In some embodiments, the side port 22 may be in fluid communication with the proximal segment and/or the delivery lumen 12.

The occlusive implant system and/or the delivery system 10 may include a core member or core wire 24 slidably and/or rotatably disposed within the delivery lumen 12 (and the proximal segment, where present). The occlusive implant system may include an occlusive implant 26, which may be configured for implantation within a left atrial appendage, releasably engaged with and/or releasably attached to a distal end of the core wire 24. In at least some embodiments, the occlusive implant 26 may be a left atrial appendage closure device. In some embodiments, a proximal end of the core wire 24 may extend proximally of a proximal end of the delivery sheath 14 and/or the proximal opening of the delivery lumen 12 for manual manipulation by a clinician or practitioner. In at least some embodiments, the delivery sheath 14 may comprise and/or may be formed from a polymeric material. In some embodiments, the delivery sheath 14 may comprise and/or may be formed from a plurality of polymeric materials. In some embodiments, the delivery sheath may comprise and/or may be formed from a combination of metallic and polymeric materials. In some embodiments, the delivery sheath 14 may include a reinforcing element, such as a mesh, a coil, a braid, etc., formed therein, embedded therein, attached thereto, etc. along at least a portion of a length of the delivery sheath 14. Other configurations are also contemplated. Some suitable, but non-limiting, examples of materials for the occlusive implant system, the core wire 24, and/or the delivery sheath 14, etc., including but not limited to metallic materials, polymeric materials, etc., are discussed below.

The occlusive implant 26 may include an expandable framework 28 (e.g., FIG. 2) configured to shift between a delivery configuration (e.g., FIG. 1), such as when the occlusive implant 26 is disposed within the delivery lumen 12 proximate the distal opening and/or within a distal portion of the delivery lumen 12, and a deployed configuration (e.g., FIG. 2) when the occlusive implant 26 is unconstrained by the delivery sheath 14.

In some embodiments, the expandable framework 28 may comprise a plurality of interconnected struts. In some embodiments, the expandable framework 28 may be compliant or semi-compliant and may generally conform to and/or be configured to sealingly engage with the shape and/or geometry of the left atrial appendage in the deployed configuration.

In some embodiments, a proximal end of the expandable framework 28 may be configured to releasably attach, join, couple, engage, or otherwise connect to the distal end of the core wire 24 (e.g., FIG. 2). In some embodiments, the proximal end of the expandable framework 28 may include a proximal hub coupled and/or non-releasably attached thereto. In some embodiments, the proximal hub may be configured to and/or adapted to releasably couple with, join to, mate with, or otherwise engage a distal end of the core wire 24. Other means of releasably coupling and/or engaging the expandable framework 28 to the distal end of the core wire 24 are also contemplated.

In some embodiments, the occlusive implant 26 may include an occlusive element 30 (e.g., a membrane, a fabric, or a tissue element, etc.) connected to, disposed on, disposed over, disposed about, or covering at least a portion the expandable framework 28. In some embodiments, the occlusive element 30 may be connected to, disposed on, disposed over, disposed about, or cover at least a portion of an outer (or outwardly facing) surface of the expandable framework 28.

In some embodiments, the occlusive element 30 may be permeable or impermeable to blood and/or other fluids, such as water. In some embodiments, the occlusive element 30 may include a polymeric membrane, a metallic or polymeric mesh, a porous or semi-porous filter-like material, or other suitable construction. In some embodiments, the occlusive element 30 prevents thrombi (e.g., blood clots, etc.) from passing through the occlusive element 30 and out of the left atrial appendage into the blood stream. In some embodiments, the occlusive element 30 promotes endothelization after implantation, thereby effectively removing the target site (e.g., the left atrial appendage, etc.) from the patient's circulatory system. Some suitable, but non-limiting, examples of materials for the occlusive element 30 are discussed below.

In some embodiments, the expandable framework 28 and/or the plurality of interconnected struts may be integrally formed and/or cut from a unitary member. In some embodiments, the expandable framework 28 and/or the plurality of interconnected struts may be integrally formed and/or cut from a unitary tubular member and subsequently formed and/or heat set to a desired shape in the deployed configuration. In some embodiments, the expandable framework 28 and/or the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the expandable framework 28 include laser cutting, machining, punching, stamping, electro discharge machining (EDM), chemical dissolution, etc. Other means and/or methods are also contemplated.

In use, the delivery sheath 14 may be advanced and/or navigated to the left atrial appendage to deliver the occlusive implant 26 thereto. In one example, the delivery sheath 14 may be advanced and/or navigated to the left atrial appendage using and/or over a guidewire. For example, the delivery sheath 14 may be advanced to the patient's left atrium and the distal end disposed adjacent to the left atrial appendage with the occlusive implant 26 disposed therein in the delivery configuration. In some embodiments, the delivery sheath 14 may include steering capability. After the distal end of the delivery sheath 14 is disposed adjacent to and/or at the left atrial appendage, the core wire 24 may be advanced distally relative to the delivery sheath 14 to advance the occlusive implant 26 out of the delivery sheath 14, where the occlusive implant 26 may shift to the deployed configuration. Exemplary occlusive implants 26 include WATCHMAN FLX™ and WATCHMAN FLX™ Pro from Boston Scientific Corporation.

While not expressly illustrated, in some embodiments, the occlusive implant system may further comprise an access device. In some embodiments, the access device may be a bi-directional steerable catheter and/or an intravascular catheter. Examples of intravascular catheters may include, but are not limited to, balloon catheters, atherectomy catheters, device delivery catheters, drug delivery catheters, diagnostic catheters, and guide catheters.

In some embodiments, the access device may be advanced and/or navigated to the left atrial appendage. In one example, the access device may be advanced and/or navigated to the left atrial appendage using and/or over a guidewire. For example, the access device may be advanced to the patient's left atrium and a distal tip disposed adjacent to the left atrial appendage. In some embodiments, the access device may include steering capability. In some embodiments, the delivery system 10 may be inserted through the access device. In some embodiments, the length of the delivery sheath 14 may be substantially equal to the length of the access device. In some embodiments, the length of the delivery sheath 14 may be slightly longer than the access device. During use, the delivery sheath 14 may be advanced within the access device with the occlusive implant 26 disposed therein in the delivery configuration. After the distal end of the delivery sheath 14 is disposed adjacent to and/or at the distal end of the access device, the core wire 24 may be advanced distally relative to the delivery sheath 14 and/or the access device to advance the occlusive implant 26 out of the delivery sheath 14 and the access device, where the occlusive implant 26 may shift to the deployed configuration.

In some embodiments, the delivery system 10, the delivery sheath 14, and/or the access device may be sized in accordance with its intended use. For example, the delivery system 10, the delivery sheath 14, and/or the access device can have a length that is in the range of about 10 to about 150 centimeters, about 25 to about 125 centimeters, about 50 to about 100 centimeters, about 25 centimeters to about 50 centimeters, about 50 to about 75 centimeters, about 75 to about 100 centimeters, etc. Other lengths are also contemplated, including but not limited to subsets of ranges disclosed herein. It is further contemplated that the outer diameter of the delivery system 10, the delivery sheath 14, and/or the access device may vary based on the use or application. In some examples, the outer diameter of the delivery system 10, the delivery sheath 14, and/or the access device may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the delivery system 10, the delivery sheath 14, and/or the access device may be a maximum of 5.66 mm (17 French) and is preferably smaller than 5.66 mm (17 French). Other configurations are also contemplated. In some embodiments, it is desirable for the outer diameter of the delivery system 10, the delivery sheath 14, and/or the access device to be as small as possible.

Catheters, catheter shafts, and/or the like, for example such as the delivery sheath 14, may be manufactured to include a number of structural features generally designed to meet the needs of such devices. For example, the delivery sheath 14 may include features such as lubricity, reinforcement (e.g., for push support, kink resistance, etc.), and radiopacity. It can be appreciated that in order to incorporate these and other features, the delivery sheath 14 may have differing components and/or sections with particular structural features. Disclosed herein are catheters, catheter shafts, and/or the like that include desirable structural features and/or manufacturing methods for producing such devices.

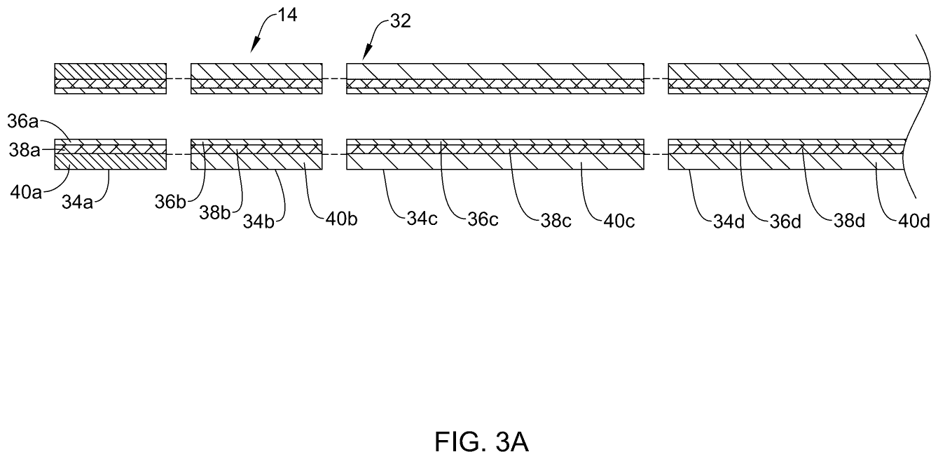

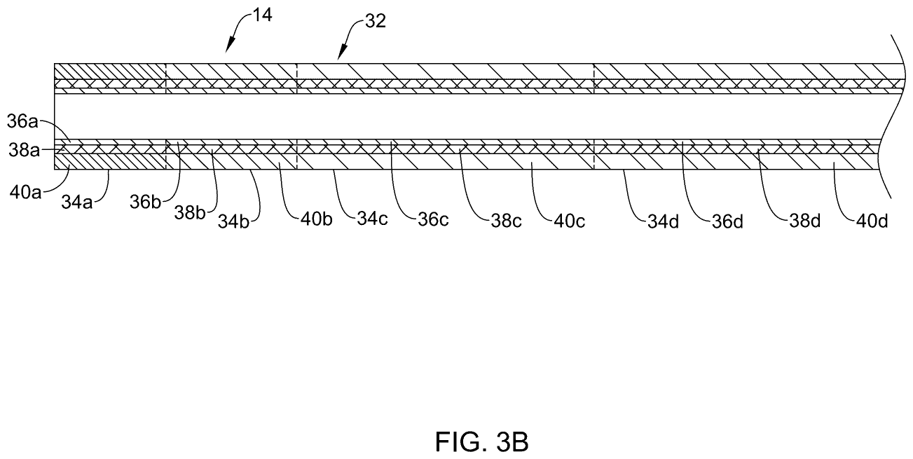

FIGS. 3A-3B illustrates the delivery sheath 14. It can be appreciated that the disclosure pertaining to the delivery sheath 14 and/or the assembly/manufacturing of the delivery sheath 14 can be utilized for a variety of catheters, catheter shafts, sheaths, and/or the like. In other words, disclosure pertaining to the delivery sheath 14 and/or other delivery sheath disclosed herein can be applied to other catheters, catheter shafts, sheaths, and/or the like without departing from the spirit of the disclosure. The delivery sheath 14 may include a sheath or shaft body 32 formed from a plurality of discrete shaft/sheath sections such as a first shaft segment 34a, a second shaft segment 34b, a third shaft segment 34c, and a fourth shaft segment 34d. While FIGS. 3A-3B illustrate four shaft segments, more or fewer shaft segments can be utilized.

In general, the shaft segments 34a, 34b, 34c, 34d are formed as separate, discrete shafts or shaft members. The shaft segments 34a, 34b, 34c, 34d may be arranged so that the shaft segment 34a is at the distal end and/or forms a distal tip whereas the shaft segment 34d may by disposed at or extend toward the proximal end. In some instances, an elongate structure may be formed using a suitable manufacturing process and the resultant structure may be used to form one or more of the shaft segments 34a, 34b, 34c, 34d. For example, a tube (e.g., an elongate tube) may be formed having the structural characteristics of the first shaft segment 34a. A section of this “pre-cursor” tube can be trimmed or cut to the desired length and the secured with one or more additional shaft segments to form a catheter or catheter shaft. Manufacturing a catheter or catheter shaft from a plurality of discrete shaft sections may be desirable for a number of reasons. For example, forming elongate pre-cursor shafts for each of the discrete segments (e.g., the shaft segments 34a, 34b, 34c, 34d) may help to control manufacturing costs as any defects observed in the pre-cursor shaft (and/or a portion of the length of the pre-cursor shaft) can be scrapped without having to scrap an entire, assembled catheter shaft. When using costly components in a catheter shaft (e.g., precious metals, radiopaque materials, etc.), such cost savings can be meaningful. Furthermore, forming a catheter shaft from a plurality of discrete segments (e.g., the shaft segments 34a, 34b, 34c, 34d) may allow for unique shafts to be formed with highly variable structural arrangements/properties. Some additional details regarding catheter shafts formed in this manner and the properties thereof are disclosed herein.

FIG. 3A is intended to show the discrete shaft segments 34a, 34b, 34c, 34d, cut to the desired length, being formed into the delivery sheath 14 by showing an exploded view of the delivery sheath 14. The assembled view of the delivery sheath 14 is shown in FIG. 3B, where the shaft segments 34a, 34b, 34c, 34d are joined together to form the delivery sheath 14. It can be appreciated that other drawings included with this disclosure may include similar views (e.g., an exploded view and an assembled view). Regardless of whether both views are shown, delivery sheaths and/or other catheter shafts disclosed herein may be understood to be manufactured using similar processes.

As shown in FIGS. 3A-3B, the first shaft segment 34a may include a plurality of layers such as an inner layer 36a, a reinforcing member or reinforcing layer 38a, and an outer layer 40a. Similarly, the second shaft segment 34b may include a plurality of layers such as an inner layer 36b, a reinforcing member or reinforcing layer 38b, and an outer layer 40b. Similarly, the third shaft segment 34c may include a plurality of layers such as an inner layer 36c, a reinforcing member or reinforcing layer 38c, and an outer layer 40c. Similarly, the fourth shaft segment 34d may include a plurality of layers such as an inner layer 36d, a reinforcing member or reinforcing layer 38d, and an outer layer 40d.

A variety of materials are contemplated for the various layers. For example, one or more of the inner layers 36a, 36b, 36c, 36d may include a lubricous material such as polytetrafluoroethylene, fluorinated ethylene propylene, and/or the like. In some instances, non-fluorinated materials such as high-density polyethylene and/or nylon may be used for one or more of the inner layers 36a, 36b, 36c, 36d. In some instances, the inner layers 36a, 36b, 36c, 36d may all be the same in material composition. In some of these and in other instances, the inner layers 36a, 36b, 36c, 36d may be substantially the same in thickness. Alternatively, the inner layers 36a, 36b, 36c, 36d may vary in material composition from one another and/or vary in thickness.

The reinforcing layers 38a, 38b, 38c, 38d may include suitable materials such as a polyether block amide, nylon, and/or other materials disclosed herein. In some instances, the reinforcing layers 38a, 38b, 38c, 38d may all be the same in material composition and/or form. For example, the reinforcing layers 38a, 38b, 38c, 38d may include a coil, a braid, a coil and a braid, a coil disposed over a braid, a braid disposed over a coil, and/or the like. Suitable materials including stainless steel, nickel-titanium alloy, polymers, and/or materials disclosed herein may be utilized. In some of these and in other instances, the reinforcing layers 38a, 38b, 38c, 38d may be substantially the same in thickness/arrangement. For example, the reinforcing layers 38a, 38b, 38c, 38d may all be closed pitch coils. Alternatively, the reinforcing layers 38a, 38b, 38c, 38d may vary in material composition, reinforcement type, thickness, arrangements, etc. Example reinforcing layers 38a, 38b, 38c, 38d may include coils, open pitch coils, closed pitch coils, metal coils, radiopaque coils, polymer coils, braids, high PIC braids, low PIC braids, metallic braids, polymer braids, braids including one or more an axially-extending wires, a coil disposed over a braid, a braid disposed over a coil, combinations thereof, and/or the like. In some instances, the reinforcing layers 38a, 38b, 38c, 38d may all include the same type of reinforcing structure. Alternatively, some of the reinforcing layers 38a, 38b, 38c, 38d may have reinforcing structures that differ from one another.

The outer layers 40a, 40b, 40c, 40d may take the form of or include a reinforcing member such as a coil, braid, combinations thereof, and/or the like. In some instances, the outer layers 40a, 40b, 40c, 40d may all be the same in material composition. The outer layers 40a, 40b, 40c, 40d may include suitable materials such as a PEBAX and/or other materials disclosed herein. In some of these and in other instances, the outer layers 40a, 40b, 40c, 40d may be substantially the same in thickness. Alternatively, the outer layers 40a, 40b, 40c, 40d may vary in material composition from one another and/or vary in thickness.

Delivery sheaths are contemplated where one or more of the shaft segments 34a, 34b, 34c, 34d omits or lacks one or more of the layers. For example, one or more of the shaft segments 34a, 34b, 34c, 34d may lack a reinforcing layer. Additionally, one or more of the various layers may include radiopaque materials. For example, one or more of the outer layers 40a, 40b may include a radiopaque marker band incorporated therein and/or include radiopaque filler.

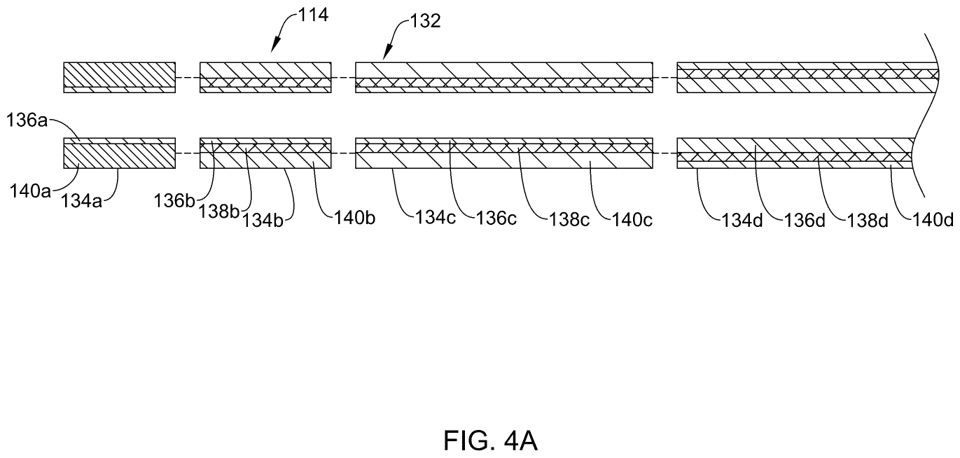

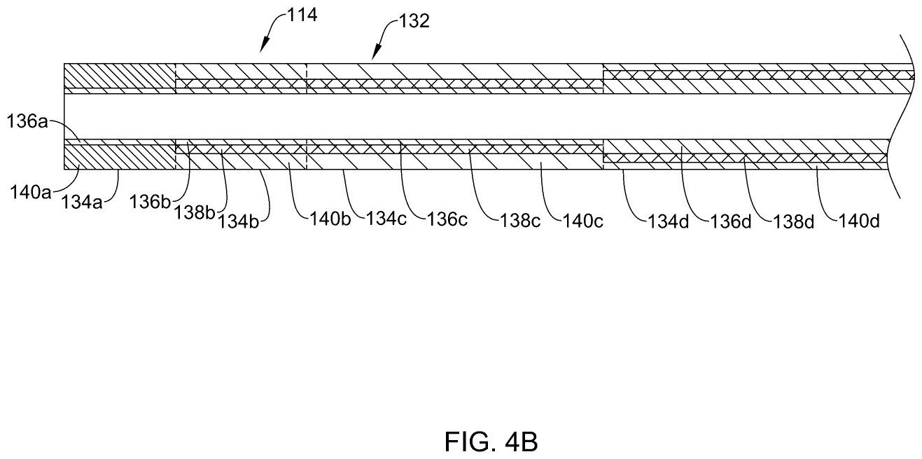

As depicted in FIGS. 3A-3B, the various layers may be substantially consistent through each of the shaft segments 34a, 34b, 34c, 34d. However, this is not intended to be limiting. For example, FIGS. 4A-4B illustrate another example delivery sheath 114, which may be substantially similar in form and function to other sheaths disclosed herein. The delivery sheath 114 may include a sheath or shaft body 132 formed from a plurality of discrete shaft/sheath sections such as a first shaft segment 134a, a second shaft segment 134b, a third shaft segment 134c, and a fourth shaft segment 134d. While FIGS. 4A-4B illustrate four shaft segments, more or fewer shaft segments can be utilized.

The first shaft segment 134a may include a plurality of layers such as an inner layer 136a and an outer layer 140a. The first shaft segment 134a may form or define a distal tip of the shaft body 132. In this example, the first shaft segment 134a may lack a reinforcing layer. The second shaft segment 134b may include a plurality of layers such as an inner layer 136b, a reinforcing member or reinforcing layer 138b, and an outer layer 140b. The second shaft segment 134b may take the form of a marker band segment. For example, the outer layer 140b may include a radiopaque material or filler. The third shaft segment 134c may include a plurality of layers such as an inner layer 136c, a reinforcing member or reinforcing layer 138c, and an outer layer 140c. Similarly, the fourth shaft segment 134d may include a plurality of layers such as an inner layer 136d, a reinforcing member or reinforcing layer 138d, and an outer layer 140d.

In the example depicted in FIGS. 4A-4B, one or more the inner layers 136a, 136b, 136c, 136d may vary in thickness. For example, the inner layer 136c may have a first thickness and the inner layer 136d may have a second thickness that is larger/thicker than the first thickness. In some of these and in other instances, one or more the outer layers 140a, 140b, 140c, 140d may vary in thickness. For example, the outer layer 140c may have a first thickness and the outer layer 140d may have a second thickness that is smaller/thinner than the first thickness.

In addition, the reinforcing layer 138c may be offset (e.g., radially offset) from the reinforcing layer 138d. This is represented in FIGS. 4A-4B (cross-sectional side views) by the longitudinal axis of the reinforcing layer 138c being positioned inward of the longitudinal axis of the reinforcing layer 138d. Radial offset may occur or otherwise be possible, for example, when layers of different thickness are used, thus resulting in the depth/radial position of the reinforcing layers 138c, 138d being different from one another.

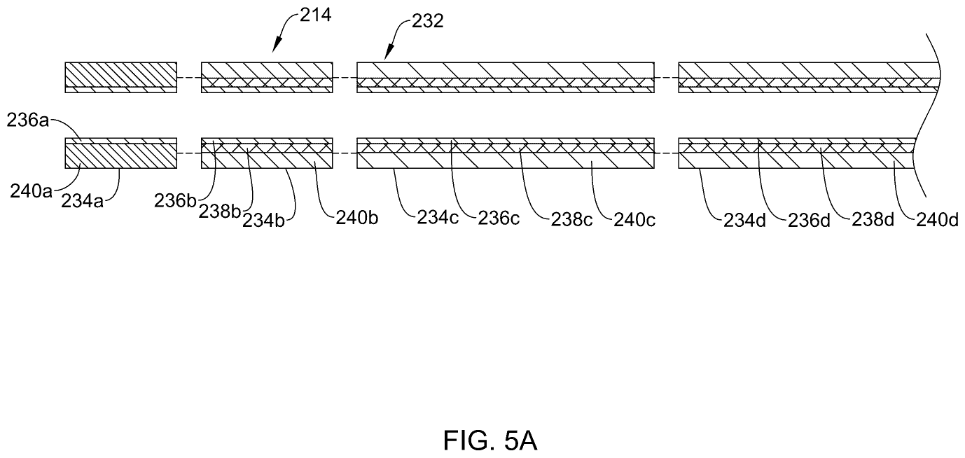

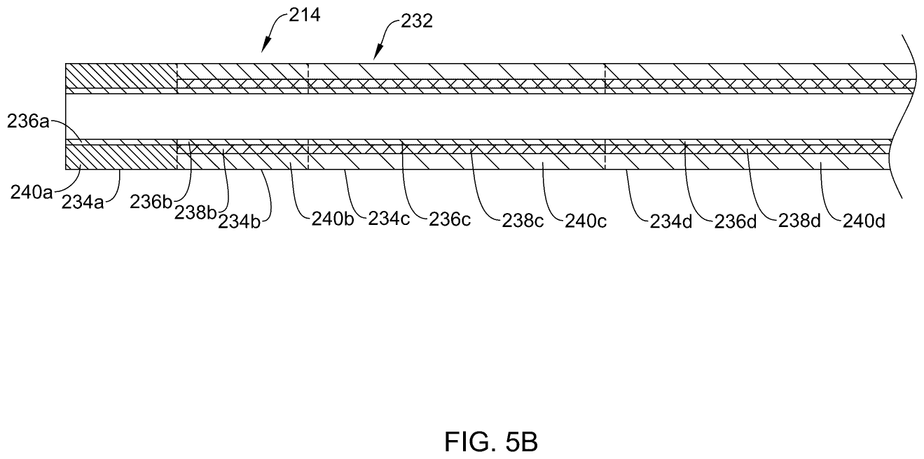

FIGS. 5A-5B illustrate another example delivery sheath 214, which may be substantially similar in form and function to other sheaths disclosed herein. The delivery sheath 214 may include a sheath or shaft body 232 formed from a plurality of discrete shaft/sheath sections such as a first shaft segment 234a, a second shaft segment 234b, a third shaft segment 234c, and a fourth shaft segment 234d. While FIGS. 5A-5B illustrate four shaft segments, more or fewer shaft segments can be utilized.

The first shaft segment 234a may include a plurality of layers such as an inner layer 236a and an outer layer 240a. The first shaft segment 234a may form or define a distal tip of the shaft body 232. The second shaft segment 234b may include a plurality of layers such as an inner layer 236b, a reinforcing member or reinforcing layer 238b, and an outer layer 240b. The second shaft segment 234b may take the form of a marker band segment. For example, the outer layer 240b may include a radiopaque material or filler. The third shaft segment 234c may include a plurality of layers such as an inner layer 236c, a reinforcing member or reinforcing layer 238c, and an outer layer 240c. Similarly, the fourth shaft segment 234d may include a plurality of layers such as an inner layer 236d, a reinforcing member or reinforcing layer 238d, and an outer layer 240d.

In some instances, the one or more of the various layers may vary through each of the shaft segments 234a, 234b, 234c, 234d. The example depicted in FIGS. 5A-5B is intended to represent differences in the reinforcing layers 238b, 238c, 238d. For example, the reinforcing layer 238b may include an open pitch coil (e.g., for enhanced flexibility) and the reinforcing layer 238c may include a closed pitch coil (e.g., for push support). Another example contemplated may include the reinforcing layer 238b including a braid and the reinforcing layer 238c including a coil.

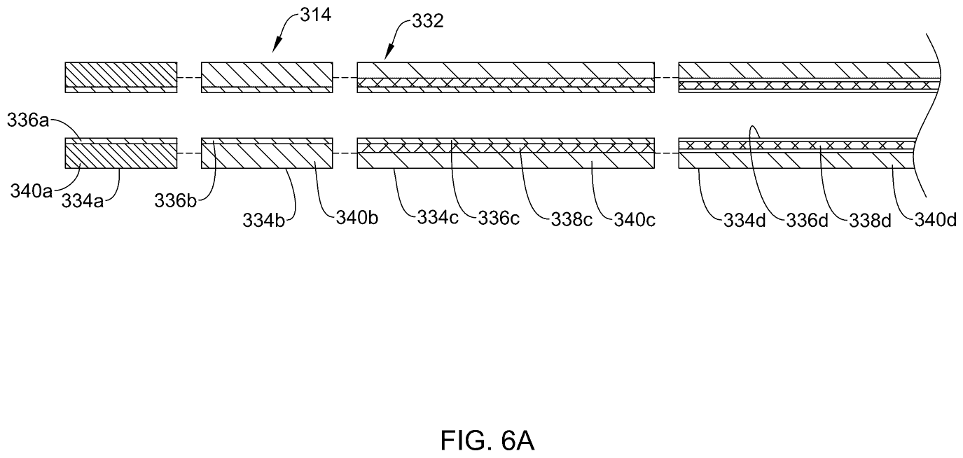

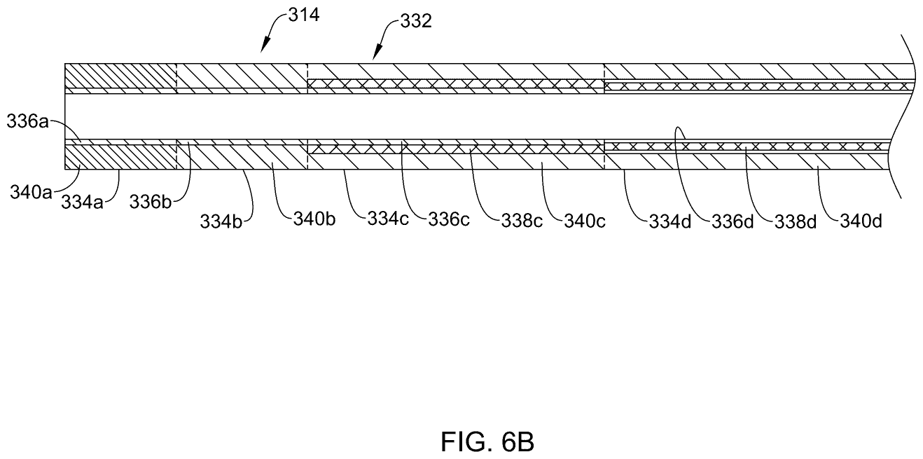

FIGS. 6A-6B illustrate another example delivery sheath 314, which may be substantially similar in form and function to other sheaths disclosed herein. The delivery sheath 314 may include a sheath or shaft body 332 formed from a plurality of discrete shaft/sheath sections such as a first shaft segment 334a, a second shaft segment 334b, a third shaft segment 334c, and a fourth shaft segment 334d. While FIGS. 6A-6B illustrate four shaft segments, more or fewer shaft segments can be utilized.

The first shaft segment 334a may include a plurality of layers such as an inner layer 336a and an outer layer 340a. The first shaft segment 334a may form or define the distal tip of the shaft body 332. The second shaft segment 334b may include a plurality of layers such as an inner layer 336b and an outer layer 340b. The second shaft segment 334b may take the form of a marker band segment. For example, the outer layer 340b may include a radiopaque material or filler. The third shaft segment 334c may include a plurality of layers such as an inner layer 336c, a reinforcing member or reinforcing layer 338c, and an outer layer 340c. Similarly, the fourth shaft segment 334d may include a plurality of layers such as an inner layer 336d, a reinforcing member 338d, and an outer layer 340d. In this example, rather than being formed as a separate layer, the reinforcing member 338d is embedded in or otherwise incorporated into the inner layer 336d of the fourth shaft segment 334d. A similar arraignment may be utilized in other shaft segments.

In at least some instances, the reinforcing layer 338c may be offset (e.g., radially offset) from the reinforcing member 338d. This is represented in FIGS. 6A-6B (cross-sectional side views) by the longitudinal axis of the reinforcing layer 338c being positioned outward of the longitudinal axis of the reinforcing member 338d. Radial offset may occur or otherwise be possible, for example, when layers of different thickness are used, thus resulting in the depth/radial position of the reinforcing layer 338c and reinforcing member 338d being different from one another.

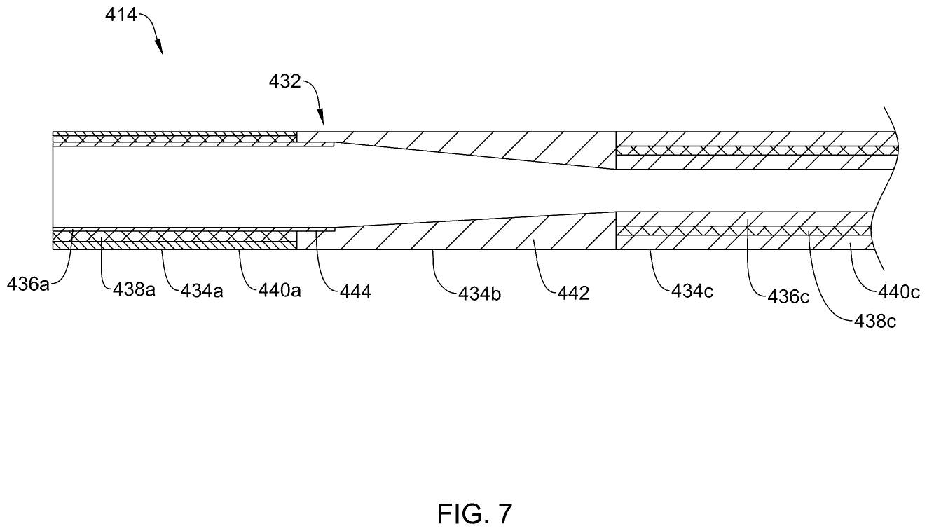

FIG. 7 illustrates another example delivery sheath 414, which may be substantially similar in form and function to other sheaths disclosed herein. The delivery sheath 414 may include a sheath or shaft body 432 formed from a plurality of discrete shaft/sheath sections such as a first shaft segment 434a, a second shaft segment 434b, and a third shaft segment 434c. While FIG. 7 illustrates three shaft segments, more or fewer shaft segments can be utilized.

The first shaft segment 434a may include a plurality of layers such as an inner layer 436a, a reinforcing member or reinforcing layer 438a, and an outer layer 440a. Similarly, the second shaft segment 434b may include one or more layers such as a layer 442. Similarly, the third shaft segment 434c may include a plurality of layers such as an inner layer 436c, a reinforcing member or reinforcing layer 438c, and an outer layer 440c.

In some instances, the one or more of the various layers may vary through each of the shaft segments 434a, 434b, 434c. For example, the inner layer 436a may include a section 444 extending along and/or overlapping with a region of the second shaft segment 434b. The second shaft segment 434b may include only a similar layer 442 that may taper such that the inner diameter of the shaft 432 may increase in the distal direction.

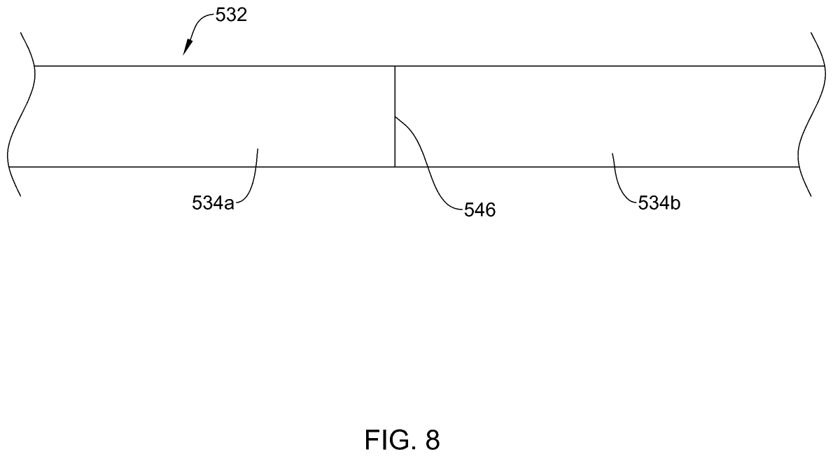

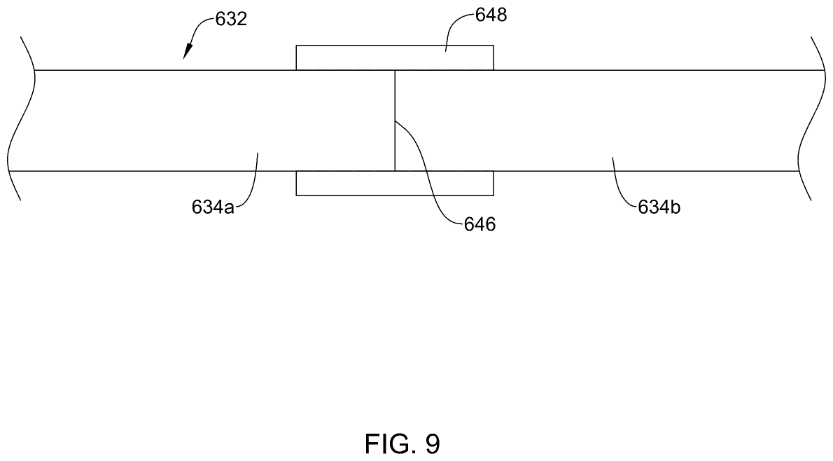

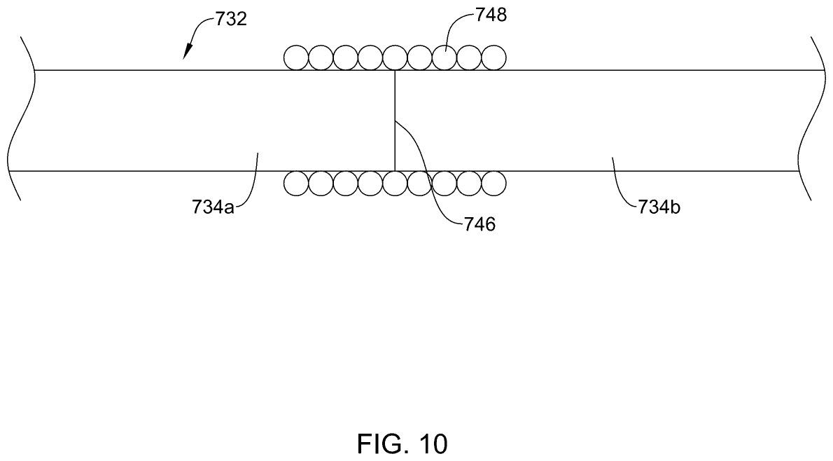

FIGS. 8-10 illustrate example arrangements and/or structures for securing together discrete shaft segments with to form a shaft. For example, FIG. 8 illustrates an example sheath or shaft body 532. In this example, a first shaft segment 534a and a second shaft segment 534b are secured together by a thermal bond, forming a joint 546. The joint 546 may be formed using a suitable thermal process such as using a hot jaw or similar fixture. In some of these an in other instances, a structure may be disposed over the joint 546 during the bonding process, which may be later removed. For example, FIG. 9 illustrates an example sheath or shaft body 632. In this example, a first shaft segment 634a and a second shaft segment 634b are secured together by a thermal bond, forming a joint 646. A joining structure such as a sleeve 648 may be disposed over the joint 646. After forming the joint 646, the sleeve 648 may be left in place or removed. In some instances, the sleeve 648 may include a heat shrink material. In some of these and in other instances, the sleeve 648 may include a radiopaque material. FIG. 10 illustrates an example sheath or shaft body 732. In this example, a first shaft segment 734a and a second shaft segment 734b are secured together by a thermal bond, forming a joint 746. A joining structure such as a coil or coil sleeve 748 may be disposed over the joint 746. After forming the joint 746, the coil sleeve 748 may be left in place or removed. In some instances, the coil sleeve 748 may include a radiopaque material.

It can be appreciated that a wide variety of arrangement are disclosed. One specific example, not intended to be limiting of a specific arrangement, may include a first segment including an inner layer formed from high density polyethylene and an outer layer formed from a 40D PEBAX material. The first segment may lack a reinforcing layer. Alternatively, the first segment may include a polymer braid reinforcing layer. The second segment may include an inner layer formed from high density polyethylene and an outer layer formed from a 40D PEBAX material loaded with a radiopaque material. The second segment may lack a reinforcing layer. Alternatively, the second segment may include a polymer braid reinforcing layer. The third segment may include an inner layer formed from high density polyethylene, a reinforcing layer including or formed from a high PIC braid, and an outer layer formed from a 55D PEBAX material. The fourth segment may include an inner layer formed from high density polyethylene, a reinforcing layer including or formed from a low PIC braid (e.g., which may have a larger diameter braid wire and/or an axially-extending wire), and an outer layer formed from a 72D PEBAX or a relatively stiff nylon material.

Another specific example, not intended to be limiting of a specific arrangement, may include a first segment including an inner layer formed from high density polyethylene and an outer layer formed from a 40D PEBAX material. The first segment may lack a reinforcing layer. Alternatively, the first segment may include a polymer braid reinforcing layer. The second segment may include an inner layer formed from high density polyethylene, a reinforcing layer including or formed from a metal braid, and an outer layer formed from a 40D PEBAX material loaded with a radiopaque material. The third segment may include an inner layer formed from high density polyethylene, a reinforcing layer including or formed from a high PIC braid, and an outer layer formed from a 55D PEBAX material. The fourth segment may lack an inner layer or include an inner layer formed from a 72D PEBAX, a reinforcing layer including or formed from a high diameter braid and coil, and an outer layer formed from a 72D PEBAX or a relatively stiff nylon material.

Another specific example, not intended to be limiting of a specific arrangement, may include a first segment including an inner layer formed from high density polyethylene and an outer layer formed from a 40D PEBAX material. The first segment may lack a reinforcing layer. Alternatively, the first segment may include a polymer braid reinforcing layer. The second segment may include an inner layer formed from high density polyethylene and an outer layer formed from a 40D PEBAX material loaded with a radiopaque material. The second segment may lack a reinforcing layer. Alternatively, the second segment may include a polymer braid reinforcing layer. The third segment may include an inner layer formed from high density polyethylene, a reinforcing layer including or formed from a high PIC braid, and an outer layer formed from a 55D PEBAX material. The fourth segment may lack an inner layer or include an inner layer formed from a 72D PEBAX material, a reinforcing layer including a 72D PEBAX or a relatively stiff nylon material, and an outer layer formed from a 72D PEBAX or a relatively stiff nylon material.

The materials that can be used for the various components of the delivery system 10 (and/or other delivery systems disclosed herein) may include those commonly associated with medical devices. For simplicity purposes, the following discussion makes reference to the delivery system 10. However, this is not intended to limit the devices and methods described herein, as the discussion may be applied to other delivery systems disclosed herein.

The delivery system 10 and/or other components of the delivery system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the delivery system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the delivery system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the delivery system 10 to achieve the same result.

In some embodiments, a degree of Magnetic Resonance Imaging (MRI) compatibility is imparted into the delivery system 10. For example, the delivery system 10, or portions thereof, may be made of a material that does not substantially distort the image and create substantial artifacts (e.g., gaps in the image). Certain ferromagnetic materials, for example, may not be suitable because they may create artifacts in an MRI image. The delivery system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for manufacturing a catheter shaft, the method comprising:
forming a first shaft member, the first shaft member including a first inner layer, a first reinforcing layer, and a first outer layer;
forming a second shaft member, the second shaft member including a second inner layer, a second reinforcing layer, and a second outer layer;
wherein at least one of (a) the first inner layer differs from the second inner layer, (b) the first reinforcing layer differs from the second reinforcing layer, and (c) the first outer layer differs from the second outer layer;
trimming the first shaft member to form a first shaft segment;
trimming the second shaft member to form a second shaft segment; and
securing the first shaft segment to the second shaft segment.

2. The method of claim 1, wherein the first inner layer is radially thicker than the second inner layer.

3. The method of claim 1, wherein the first reinforcing layer includes a first braid and wherein the second reinforcing layer includes a second braid different from the first braid.

4. The method of claim 1, wherein the first reinforcing layer includes a braid and wherein the second reinforcing layer includes a coil.

5. The method of claim 1, wherein the first reinforcing layer includes a metal and wherein the second reinforcing layer includes a polymer.

6. The method of claim 1, wherein the first reinforcing layer is radially offset from the second reinforcing layer.

7. The method of claim 1, wherein the first outer layer is radially thicker than the second outer layer.

8. The method of claim 1, wherein securing the first shaft segment to the second shaft segment includes securing the first shaft segment to the second shaft segment with an adhesive bond.

9. The method of claim 1, wherein securing the first shaft segment to the second shaft segment includes securing the first shaft segment to the second shaft segment with a thermal bond.

10. The method of claim 1, wherein securing the first shaft segment to the second shaft segment includes securing the first shaft segment to the second shaft segment with a joining structure.

11. A medical device, comprising:
an elongate shaft formed from a plurality of discrete shaft segments that are joined together, the plurality of discrete shaft segments including a first shaft segment and a second shaft segment;
wherein the first shaft segment includes a first inner layer, a first reinforcing layer, and a first outer layer;
wherein the second shaft segment includes a second inner layer, a second reinforcing layer, and a second outer layer; and
wherein at least one of (a) the first inner layer differs from the second inner layer, (b) the first reinforcing layer differs from the second reinforcing layer, and (c) the first outer layer differs from the second outer layer.

12. The medical device of claim 11, wherein the first reinforcing layer includes a first braid and wherein the second reinforcing layer includes a second braid different from the first braid.

13. The medical device of claim 11, wherein the first reinforcing layer includes a braid and wherein the second reinforcing layer includes a coil.

14. The medical device of claim 11, wherein the first reinforcing layer includes a metal and wherein the second reinforcing layer includes a polymer.

15. The medical device of claim 11, wherein the first reinforcing layer includes a tungsten braid and wherein the second reinforcing layer includes a stainless steel braid.

16. The medical device of claim 11, wherein the first reinforcing layer is radially offset from the second reinforcing layer.

17. The medical device of claim 11, further comprising a third shaft segment coupled to the second shaft segment.

18. The medical device of claim 17, wherein the third shaft segment is free of a reinforcing layer.

19. The medical device of claim 17, wherein the third shaft segment includes a radiopaque marker.

20. A method for manufacturing a catheter shaft, the method comprising:
forming a first shaft segment, the first shaft segment including a first inner layer, a first outer layer, and a first reinforcing member disposed between the first inner layer and the first outer layer;
forming a second shaft segment, the second shaft segment including a second inner layer, a second outer layer, and a second reinforcing member disposed between the second inner layer and the second outer layer;
wherein at least one of (a) the first inner layer differs from the second inner layer, (b) the first outer layer differs from the second outer layer, and (c) the first reinforcing member differs from the second reinforcing member;
trimming a first pre-determined length of the first shaft segment to form a first shaft segment;
trimming a second pre-determined length of the second shaft segment to form a second shaft segment; and
securing the first shaft segment to the second shaft segment.
